(19) United States
(12) Patent Application Publication
(10) Pub. No.: US 2003/0187311 A1
(43) Pub. Date: Oct. 2, 2003

(54) Method of separating and converting hydrocarbon composites and polymer materials

(76) Inventor: James P. Barvincak, Ankeny, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/112,322

(22) Filed: Mar. 29, 2002

(51) Int. Cl. C07C 1/00

(52) U.S. Cl. 585/240; 585/241; 585/242

(57) ABSTRACT

A novel method of separating hydrocarbon and/or polymer from waste and natural materials is described herein. The method first involves removing the moisture from the material containing hydrocarbon and/or polymer, then reacting the material with a catalyst in order to separate the hydrocarbon and/or polymer from the material. The drying and catalytic reaction steps preferably take place under a slightly negative pressure and substantially in the absence of oxygen. The hydrocarbon/polymer is then recovered in the form of a vapor or gas. The material, substantially free of hydrocarbons and polymers may be further processed, recycled, or safely disposed of.

### FIELD OF THE INVENTION

This invention relates to a safe and efficient method of separating hydrocarbon composites and converting polymers to hydrocarbons from waste, waste products, and natural materials containing hydrocarbon composites and polymers.

### BACKGROUND OF THE INVENTION

Wastes containing hydrocarbon composites and polymers are plentiful, and becoming a growing hazard. There are numerous sources of hydrocarbon waste materials, including pump sand, invert mud, tailing ponds, and refinery wastes.

Pump sand consists of the small amount of sand that is pumped out of the ground along with crude oil. After separation, the pump sand contains 5-10% crude oil plus 2-5% water. While this amount is only about 2 to 3 pounds pump sand per barrel of oil, in view of the fact that oil companies pump as much as 200,000 barrels of crude oil per day, the amount of pump sand is considerable. For instance, the cost of storing a ton of pump sand typically ranges between $100-$150/ton. Thus, oil companies can spend up to $50,000-$60,000 per day just for storage of their pump sand.

Another type of hydrocarbon waste material is invert mud. When drilling for gas or oil, the drilling rigs pump a mixture of clay, water, and diesel fuel into the hole to keep it from collapsing. This mixture is recycled until the well is completed. The mud left over, i.e. the “invert mud”, is difficult to reclaim. It is often put into ponds, tanks, or salt mines to be removed at a later time. The cost of storing this invert mud is expensive, ranging from $100-$150/ton.

A tailing pond is the waste created from processing oil sands. While today's processes are more efficient and of less concern, early processes created several “lakes” of tailing ponds estimated at 400 acres and 40 feet deep that will have to cleaned up in the future. It is estimated that by the year 2025 Syncrude will have produced an estimated one billion cubic meters of fine tailings.

Oil refineries in the Houston area alone produce at least 240 tons/day of waste consisting of tank bottoms, crude oil spills/dirt, and tower wastes. At present, these refineries haul their waste to a disposal site in Louisiana at a cost of approximately $600/ton. The annual waste disposal costs for these refineries are nearly $50 million per year.

Hydrocarbon wastes are produced in every industrialized country around the world where they are often stockpiled in legal and illegal landfills. Some hydrocarbon waste materials are disposed of using inefficient and contaminating methods, while only a small percentage of these waste materials are reclaimed and reused.

Incinerating, solvent extraction, thermal desorbers, and burying are currently the most common means of hydrocarbon waste disposal. These methods, however, are very expensive and result in pollution of the environment. These methods further only result in a postponement of the waste disposal problem, rather than a permanent solution.

Another common method of hydrocarbon waste disposal involves the use of centrifugation for cleanup of fuel oil tank bottoms, refining waste, pump sands, invert mud, and land/oil spills. Centrifugation involves the continuous separation of solid and liquid waste materials which are then discharged separately. The solids discharged from the centrifuge, however, always have a residual liquid remaining. In the case of water, oil, and “dirt”, the solids remaining (the centrifuge “cake”) can have as high as 7% oil residual. In the past, this centrifuge cake has been left at the waste removal site or disposed of in a landfill. In view of the environmental concerns of today, such disposal options are no longer acceptable. The current options for disposal of the centrifuge cake are storage, high temperature desorbers, or incineration. All of these methods are very expensive, however, and result in little or no recovery of usable hydrocarbon by-products.

Other hydrocarbon waste disposal processes involve “cracking” the polymers and hydrocarbon deposits. Cracking is a process whereby heavy hydrocarbon molecules are broken up into lighter molecules by means of heat and usually pressure (thermal cracking), and sometimes involves the use of catalysts (catalytic cracking). These cracking methods, however, involve the use of extremely high temperatures, addition of hydrogen and pressures which are expensive, hazardous to operate, and do not result in a complete recovery of the non-waste components.

Tar sands and oil shale comprise a major natural source of hydrocarbons. With the declining availability and rising price of conventional oil, tar sands and oil shale have enormous commercial potential. Tar sands are grains of sand intimately associated with a heavy, distillate residue known as bitumen. Tar sands contain about 7-15% bitumen which has an API gravity of about 8° and, thus, are similar to heavy crude. Conventional processing of tar sands includes strip mining and extracting with a hot water process that separates the bitumen from the minerals in the rock matrix. This processing method requires a ton of water per ton of tar sand. Disposal of the solid waste of about 10 tons per ton of sand oil produced is problematic, and a considerable environmental concern.

Oil shales are inorganic rocks that contain organic matter, mostly kerogen but some bitumen can also be present. Oil shales can contain greater than 50% organic matter by weight, or up to 150 gallons of oil per ton of rock. In fact, it has been determined that the Green River formation in the adjoining corners of Colorado, Utah, and Idaho could potentially supply the United States with fuel for 339 years at 1984 consumption levels.

Oil shales are often extracted using a retorting method wherein the shale is crushed and heated to approximately 500° C. with steam and the evolved liquid and gaseous products are collected. Retorting can also be performed in situ by drilling two wells into an underground tunnel where explosions reduce the shale to rubble. In this method, steam is pumped into one well and the retorted oil is pumped up from the other. Environmental problems associated with these extraction methods include difficulty in disposing of the light fluffy ash by-product, as well as the necessity of using large quantities of water in order to process the shale.

The present inventor has now discovered a novel means of separating hydrocarbon composites and polymers from waste, waste products, and natural materials, such as tar sands and oil shale. This novel processing method allows for the collection of heavy oils, lights oils, and gaseous hydrocarbons from waste and natural sources, leaving a clean residue in the form of soil, carbon, metals and ash, etc. which may be further processed into usable byproducts. The recovered hydrocarbon can be further processed or burned for process heat or co-generation of steam and electricity.

Accordingly, it is a primary objective of the present invention to provide a novel method and means of processing and recycling hydrocarbon and/or polymer waste materials.

It is also a primary objective of the present invention to provide a novel method and means of processing and recycling hydrocarbons and/or polymers from natural sources, including tar sands and oil shales.

It is a further objective of the present invention to provide a novel method and means of processing and recycling hydrocarbons and/or polymers that is clean and efficient.

It is a further objective of the present invention to provide a novel method and means of processing and recycling hydrocarbons and/or polymers that results in more complete recovery of non-waste components than previous methods.

It is still a further objective of the present invention to provide a novel method and means of processing and recycling hydrocarbons and/or polymers that is less expensive than conventional methods.

It is yet a further objective of the present invention to provide a novel method and means of processing and recycling hydrocarbons and/or polymers that does not require the use of high temperatures or pressures.

It is still a further objective of the present invention to provide a novel method and means of processing and recycling hydrocarbons and/or polymers that is not hazardous.

It is a further objective of the present invention to provide a novel method and means of processing and recycling hydrocarbons and/or polymers that does not require the controlled disposal of waste by-products.

The method and means of accomplishing each of the above objectives as well as others will become apparent from the detailed description of the invention which follows hereafter.

### SUMMARY OF THE INVENTION

The present invention describes a method of processing and recycling hydrocarbons and/or polymers from waste materials and natural sources. The method is less expensive, less hazardous to operate, and results in better recovery of hydrocarbons and polymers than prior art methods.

The method involves placing hydrocarbon and/or polymers bearing materials in a heated vessel depleted of most of its oxygen, preferably under a slight negative pressure. Once the moisture in the material is reduced, the material is reacted with a refinery catalyst at a temperature ranging between about 110-227° C. The catalyst causes the polymer or hydrocarbon to “crack”, and the temperature of the material to increase without the input of additional energy. During this reaction, hydrocarbon and converted polymers are preferably vaporized and drawn off by a vacuum. The processed material, substantially free of hydrocarbon and polymer is then allowed to cool. The heat recovered from the material may be further processed or recycled for use in the initial heating step of this process.

### BRIEF DESCRIPTION OF THE DRAWINGS

FIG. 1 is a flow chart showing a preferred hydrocarbon reclamation process of the invention.

FIG. 2 is a diagram of a preferred system for processing hydrocarbons in accordance with this invention.

FIG. 3 is a cross section of the preferred system of processing hydrocarbons taken along line AA of FIG. 2.

FIG. 4 illustrates preferred feed systems for treatment of various types of raw materials in accordance with the processes of the invention.

### DETAILED DESCRIPTION OF THE PREFERRED EMBODIMENT

The present invention relates to the development of an efficient, safe, and clean method of separating hydrocarbon and polymer composites from waste, waste products, and natural materials. The procedure involves the placement of hydrocarbon composites and polymer-bearing materials along with a catalyst in a heated vessel depleted of most of its oxygen, preferably under a slight negative pressure, to create a reaction that allows the hydrocarbon and/or polymer components to be separated from the material in the form of gas or vapors.

The invention is useful for treating any waste or natural material that contains hydrocarbons or polymers. Examples of hydrocarbon and polymer waste products include, but are not limited to, pump sand, tailing ponds, refinery wastes, soil contaminated by oil field wastes, tank bottoms, slops, invert or drilling mud, rubber from waste tires, hoses, gaskets, and belts, used oil filters, naturally occurring oil-bearing soil, tar sands, plastics, oil sands, coals, oil absorbents, and creosote. The invention is also useful in extracting hydrocarbons from natural sources, such as tar sands and oil shale.

The process first involves removing moisture from the hydrocarbon and/or polymer-bearing materials. This is preferably accomplished by drying the materials by placing them in a vessel and heating to a temperature ranging between about 80-150° C. for a time period sufficient to reduce the moisture content of the materials, preferably to a moisture content of about 1-10% by weight. The temperature is most preferably about 110° C. In general, the higher the concentration of moisture in the material, the higher the preferred processing temperature. Persons skilled in the art will appreciate that the materials may be dried at higher or lower temperatures for shorter or longer periods of time, respectively, to achieve the requisite moisture content. Prior to placement in the vessel, the waste materials are preferably reduced in size and any excess moisture is removed, typically by centrifugation.

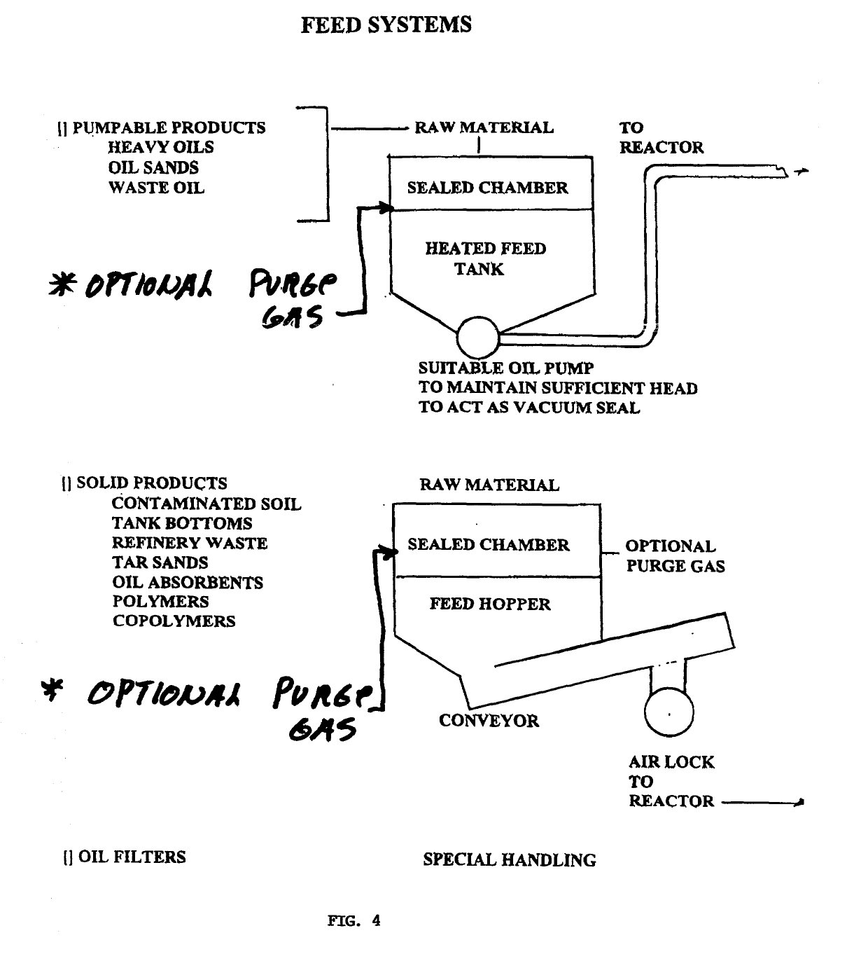

FIG. 4 illustrates preferred feed systems for the raw materials processed in the invention. As shown, the process used to handle the materials depends on the nature of the materials. For instance, pumpable products, such as heavy oils, oil sands, and waste oil are first placed in a sealed chamber that keeps the vacuum in the system and oxygen out. The materials are next transferred to a heated feed tank, then fed through an oil pump that maintains sufficient head to act as a vacuum seal. Other raw materials, such as oil filters, require special handling. Specifically, oil filters need to be crushed and/or shredded before being fed to a special belt-style conveyor for processing because of the large volume of metal present.

The material is preferably pretreated in various manners depending on the nature of the raw material. For instance, hydrocarbon contaminants, such as oil spills, soil, refinery waste, and storage tanks are preferably screened, the material washed and allowed to settle, then separated via centrifugation. Natural oil sources such as heavy oils, oil sands, pump sands, and tar sands are preferably preheated to a temperature of about 150-250° C., the free water separated via centrifugation or evaporation, and the material ground and sized. Recyclables, such as waste oil, oil absorbents, and oil filters, are preferably preheated to a temperature of about 150-250° C. Man-made materials such as tires and plastics are preferably shredded and sized prior to processing.

The moisture in the hydrocarbon and polymer-containing materials may also be removed using other conventional means known in the art including, but not limited to, centrifugation and air drying. Persons skilled in the art can readily appreciate such additional methods. It is generally preferred to centrifuge the materials prior to processing if they contain 10% or more moisture by weight.

During the drying process, moisture is preferably removed from the material as a vapor or gas. This is preferably accomplished by placing the material under a slightly negative pressure (i.e. a vacuum) ranging from about 20 to 250 mm Hg in order to encourage the flow of vapor. The preferred pressure is about 100 mm Hg. As used herein, the term “slightly negative pressure” refers to any pressure that is less than neutral pressure but not greater than about 250 mm Hg. The vapor flow can also be enhanced without the use of a vacuum (i.e. at neutral or positive pressure) through the use of a purge gas.

Thus, moisture is preferably removed from the waste material by elevating its temperature which drives the moisture from the material in the form of water vapor. This water vapor is preferably removed by a vacuum generator which draws the vapor through a condenser where the vapor is liquefied.

The drying process also preferably takes place in an atmosphere that is substantially free of oxygen in order to prevent the combustion of methane, propane, butane, and other hydrocarbon by-products. As used herein, the term “substantially free of oxygen” refers to an atmosphere that contains an insufficient amount of oxygen to cause an explosion during the processing of the hydrocarbons and/or polymers in accordance with this invention.

Once the moisture level of the hydrocarbon/polymer-containing material is reduced to an acceptable level, the material is reacted with at least one refinery catalyst at an elevated temperature which causes the hydrocarbon and/or polymer waste to “crack”. Examples of suitable catalysts for use in this invention include any catalysts typically used in refinery cracking methods including, but not limited to, catalysts containing zeolite, aluminum hydrosilicate, bauxite, bentonite, Fullers earth, and/or silica-alumina. Such catalysts are well known in the art. Preferred catalysts include those from the montmorillonite clay family, with or without metals incorporated, such as nickel, molybdenum, cobalt, tungsten, iron, palladium, rhenium, tin, magnesium, and vanadium. The catalyst is added to the material in an amount of 1-10% by weight of the material, with about 3% by weight of the material being preferred. The material is preferably reacted with the catalyst at a temperature ranging between about 110-227° C. During the reaction process, the catalyst and material are preferably agitated.

Instead of actually mixing the catalyst with the material, the material may be reacted with the catalyst by placing the catalyst next to or in close proximity to the material in the same concentration described above. The catalyst and material must be sufficiently close in proximity to allow the compounds to react. This distance will primarily depend on the temperature of the reaction and the type of catalyst used.

As noted above, the catalyst causes the hydrocarbon and/or polymer-containing material to “crack” and separate polymers and hydrocarbon composites from the material being processed. The cracking process causes a reduction of long-chain hydrocarbons, organic material, and polymers to a lower boiling range and short-chained hydrocarbons.

The catalytic “cracking” reaction usually increases the temperature of the waste material by about 100-200° C., or to about 350-450° C. (depending on the rate of removal of waste vapors) without the input of additional heat or energy into the process. As with the drying step, the catalytic reaction preferably takes place at a negative pressure ranging from about 20-250 mm Hg, with about 100 mm Hg being preferred. Again, the reaction can take place at atmospheric pressure or a slightly positive pressure with the use of a purge gas, as already described in detail above. Further, the catalytic reaction preferably takes place in a substantially oxygen-free atmosphere.

It should be appreciated that the above-referenced drying and catalytic reaction steps may take place simultaneously or separately.

The catalytic reaction is preferably allowed to continue until all hydrocarbon/polymer vapors and gases are driven from the starting material. The vapors or gases produced from the drying and catalytic reactions are separated from the material being processed, and may be condensed in a cooling or distillation tower as a liquid or liquefied gas. Some of the vapor is in C₁-C₅ gaseous form and will not condense. This gaseous vapor that will not condense is either collected and sold as surplus gas or burned as fuel to support the process. The recovered hydrocarbon/polymer oils and gases can be further processed into separation fractions of use in the generation of steam and/or electricity.

The recovered material is allowed to cool, and then may be recycled in other industrial processes or safely discarded. The degree to which the polymers and hydrocarbons are removed from the starting material varies and depends on the type of hydrocarbons/polymers recovered from the material, the EPA standards on acceptable levels of polymer/hydrocarbon-free materials, and the degree of removal of hydrocarbon/polymer desired by the customer.

The heat given off during the material's cooling process can be recovered and used to heat or dry the waste material at the beginning of this process. The substantially waste-free material often contains recoverable minerals, metals, carbon, or dirt suitable for construction fill.

Persons skilled in the art will readily understand that the processes described above may be performed in a one-step process, or in several steps. For instance, as already noted, the drying and catalytic steps may occur simultaneously, or take place in subsequent steps. While the drying and catalytic steps are occurring, the waste vapors may also be simultaneously removed and condensed.

In the alternative, the process of this invention may take place in several steps and in numerous chambers or containers in a factory or manufacturing process. For instance, the drying step may take place in a first chamber, the catalytic reaction step in a second chamber, and the separation step in a third chamber. Persons skilled in the art will also readily appreciate that the processes of this invention may be accomplished using a variety of equipment and techniques that are well known in the art, including conveyor belts, chambers, condensers, centrifuges, distillers, vacuum generators, etc. The specific equipment and processes used are not crucial so long as the intended result is accomplished.

The following examples are offered to illustrate but not limit the invention. Thus, they are presented with the understanding that various formulation modifications as well as method of delivery modifications may be made and still be within the spirit of the invention.

### EXAMPLE 1

### Preferred Hydrocarbon/Polymer Reclamation Process

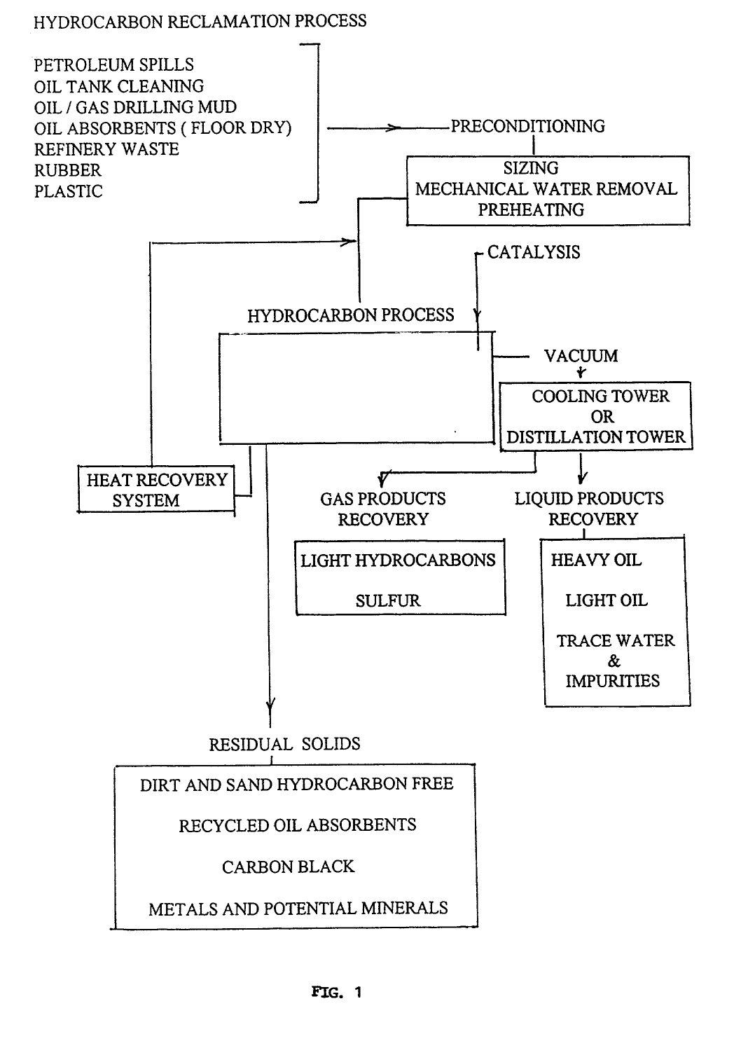

FIG. 1 illustrates a preferred hydrocarbon reclamation process in accordance with the present invention. Hydrocarbon and polymer waste materials, such as petroleum spills, oil tank cleaning, oil/gas drilling mud, oil absorbents, refinery waste, rubber, or plastic, as well as natural materials such as tar sand, oil sand, and heavy crude, are preconditioned by (a) reducing particle size; (b) removing excess water; and (c) heating.

The heated material next undergoes the catalytic reaction, and enters the “hydrocarbon process”. There, the waste gases and vapors are removed by vacuum and enter a cooling or distillation tower. The gaseous products are recovered in the form of light hydrocarbons, while the liquid products are recovered as heavy oils, light oils, trace water, and various impurities.

The material with the hydrocarbon/polymer removed is recovered and cooled. The heat given off by the material during the cooling process may be recovered and recycled for use in the heating and/or catalytic reactions. The residual solids, substantially free of hydrocarbon wastes and polymers, are comprised primarily of dirt, sand, recycled oil absorbents, carbon black (from rubber), metals, and minerals that may be recycled and used in other processes, or safely discarded.

### EXAMPLE 2

### Preferred Hydrocarbon/Polymer Reclamation Process

1. The mixture or compound of materials making up the hydrocarbon composites or polymer-bearing waste is reduced in size, conditioned, excess moisture removed by centrifugation, and fed into a machine line.

2. The material is fed into the first of three heated chambers equipped with internal conveyors and airlocks to support a normal negative operating pressure of 100 mm Hg and low presence of oxygen.

3. Once conveyed into the first chamber, moisture is removed by heating the mixture to a temperature of about 110° C. This elevated temperature drives the moisture from the mixture in the form of water vapor that is removed by the vacuum generator. This in turn draws the vapor through a condenser wherein the vapor is liquefied. The liquid is clarified through a centrifuge if necessary prior to disposal.

4. The dried material passes through an airlock and into a second heated chamber that supports a negative pressure of about 100 mm Hg. The chamber is equipped with a conveyor to move the material and provide agitation.

5. A refinery catalyst is added to the material in the second chamber and/or placed in a tray in close proximity to the material. The temperature of the material is raised to between 110-227° C., whereby the catalyst causes a reaction to take place with the hydrocarbon composites and polymers. This reaction, called cracking, raises the temperature of the mixture to about 371° C., depending on the rate that waste vapors are removed from the chamber and the type of hydrocarbon and/or polymer being removed. During the cracking reaction, hydrocarbon composites and converted polymers are vaporized and drawn off by the vacuum generator maintaining the negative pressure in the chamber.

6. The vapors are drawn through a condenser where they are condensed and collected. Some of the vapor is in the gas form and will not condense in the condenser. This gaseous vapor is either collected or burned as fuel to support the process. A small amount of the gas vapor or a small amount of inert gas can be introduced back into the chamber as purge gas to promote the removal of vapors being swept from the chamber.

7. Material conveyed through the second chamber is transferred into a third chamber equipped with a conveyor, heated to a temperature of about 215° C., and under a negative pressure of about 100 mm Hg. The catalytic reaction that began in the second chamber continues in the third chamber until all of the vapors have been driven and collected from the material. The vapors removed by the vacuum generator are treated the same as they were in the second chamber.

8. The processed material, free of hydrocarbon composites and polymers at the outlet end of the third chamber, is expelled through an airlock and allowed to cool. Some of the heat given off by the cooling mass of material can be used to warm material being prepared at the beginning of the process. Optional chambers may be added to handle a high volume of hydrocarbons or polymers if necessary. An additional chamber is used for a heat recovery system.

### EXAMPLE 3

### Preferred Equipment for Use in the Hydrocarbon/Polymer Reclamation Process

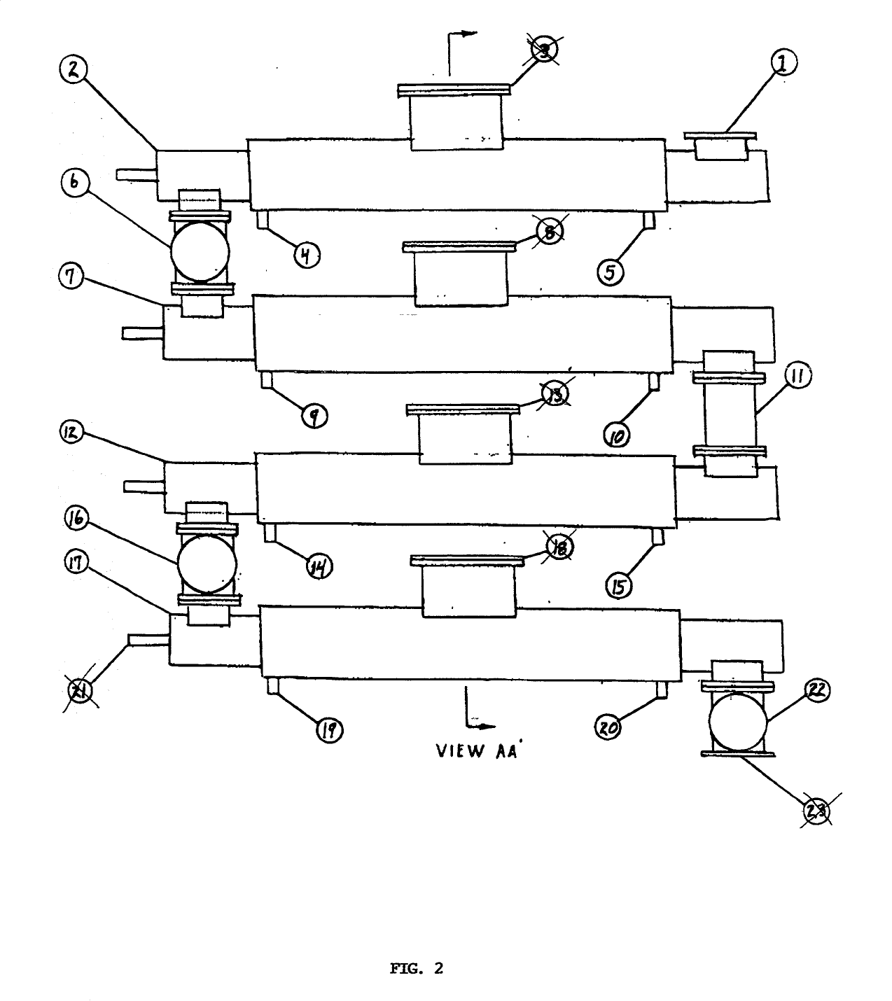

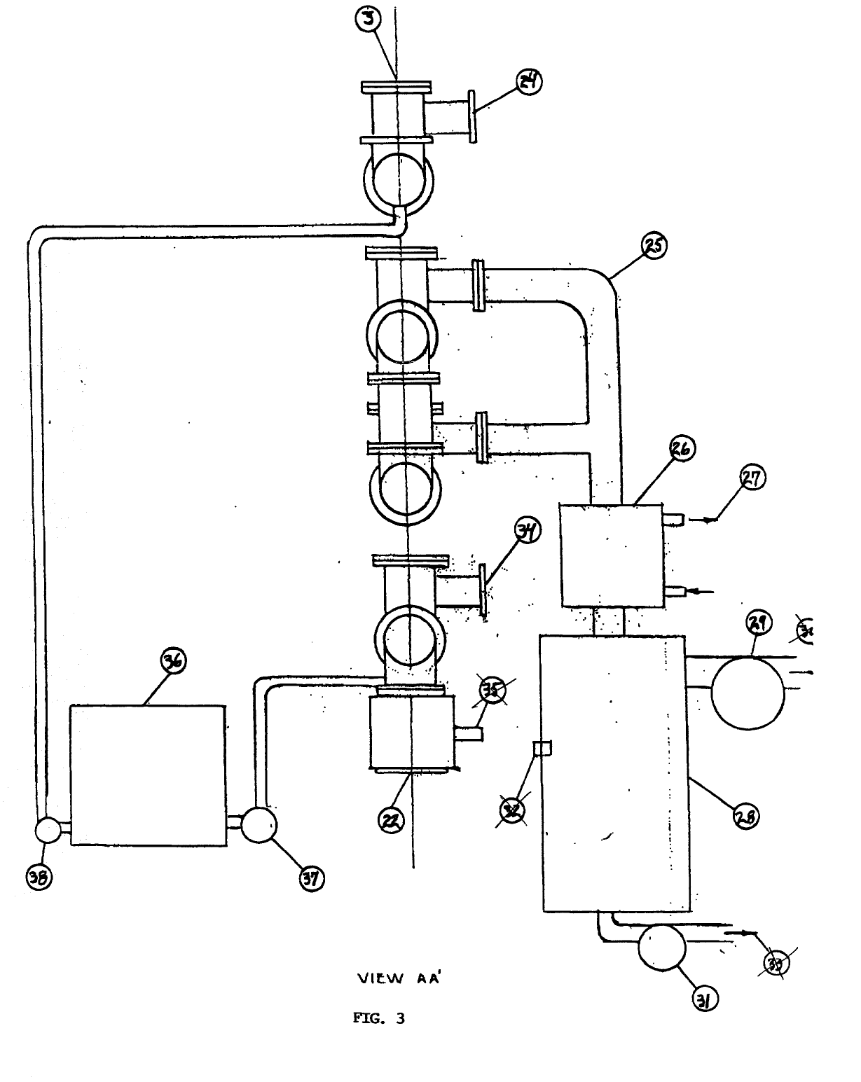

FIG. 2 and FIG. 3 illustrate preferred equipment for use in the hydrocarbon/polymer reclamation process of this invention.

Natural and/or waste material containing hydrocarbon and/or polymer is fed into the raw material inlet 1 where it proceeds to the first stage chamber, preheat, and water removal area 2. There, the material is heated using hot oil inlet 4 and dried to reduce its moisture content. Numeral 5 indicates the hot oil discharge area. Other hot oil inlets are indicated by numerals 9, 14, and 19, while other hot oil discharge areas are indicated by numerals 10, 15, and 20. Airlock 6 provides an atmosphere substantially free of oxygen. Other airlocks are indicated by numerals 16 and 22. Water vapor produced during the drying stage is removed through a water vapor discharge 24.

The dried material next enters the second stage chamber 7 wherein it is reacted with a catalyst. Once the reaction is complete, the material is transferred through a transfer chute 11 to a third stage chamber 12 for providing additional heat and retention time to further separate the polymers/hydrocarbons from the material if necessary. Hydrocarbon/polymer vapors produced in the second and third stage chambers 7 and 12 are removed via hydrocarbon vapor discharge 25. The vapors enter a condenser 26 fed by cooling medium 27. The condensed hydrocarbons/polymer enter condensed oil chamber 28 and are removed for further processing via pump 31. Noncondensed hydrocarbons/polymers are removed from the system with a vacuum generator 29.

Heat from the earlier stages of the process may be recovered in a final stage 17 and recovered via pump 37. Excess heat is exhausted via hot air exhaust 34. The heat then enters hot oil recovery system 36 and is recycled via pump 38 to heat oil in the first stage chamber 2.

What is claimed is:

1. A method of separating hydrocarbon and/or polymer composites from natural and waste materials comprising: drying a material containing hydrocarbon and/or polymer to form a dried material; reacting the dried material with at least one catalyst for a time period sufficient to separate the hydrocarbon and/or polymer from the dried material.

2. The method of claim 1 wherein the hydrocarbon and/or polymer is separated as a substance selected from the group consisting of a liquid, a gas, and a by-product.

3. The method of claim 1 wherein the material is dried until the moisture content of the material is 1-10% by weight or less.

4. The method of claim 1 wherein the drying step comprises heating the material to a temperature ranging from about 110-227° C.

5. The method of claim 1 wherein the drying step takes place at a negative pressure of from about 20-100 mm Hg.

6. The method of claim 5 wherein the drying step takes place at a negative pressure of about 100 mm Hg.

7. The method of claim 1 wherein the drying step takes place substantially in the absence of oxygen.

8. The method of claim 1 wherein the material is reacted with the catalyst at a temperature ranging from about 110-227° C.

9. The method of claim 1 wherein the material is reacted with the catalyst at a negative pressure of about 100 mm Hg.

10. The method of claim 1 wherein the material is reacted with the catalyst substantially in the absence of oxygen.

11. The method of claim 1 wherein the material is reacted with at least one catalyst selected from the group consisting of zeolite, aluminum hydrosilicate, bauxite, bentonite, Fullers earth, and silica-alumina.

12. The method of claim 1 wherein the material is reacted with at least one catalyst from the montmorillonite clay family.

13. The method of claim 1 wherein the hydrocarbon and/or polymer is separated from the material in the form of vapor or gas.

14. The method of claim 13 wherein the hydrocarbon and/or polymer is separated from the material in the form of vapor or gas through the use of a cooling tower or distillation tower.

15. The method of claim 1 wherein the recovered material is dried.

16. The method of claim 15 wherein heat produced in drying the recovered material is recovered and used in the drying step.

17. The method of claim 1 wherein the drying and reacting steps are performed simultaneously.

18. The method of claim 1 wherein the drying and reacting steps are performed separately.

19. A method of separating hydrocarbon and/or polymer composites from materials comprising: drying a material containing hydrocarbon and/or polymer until the moisture content of the material is 1-10% by weight or less to form a dried material; reacting the dried material with a catalyst for a time period sufficient to separate the hydrocarbon and/or polymer from the dried material to form product and recovered material; and collecting the product from the recovered material; wherein the drying and reacting steps are performed under a slightly negative pressure.

20. The method of claim 1 wherein the drying and reacting steps are performed substantially in the absence of oxygen.

21. A method of separating hydrocarbon and/or polymer composites from and/or polymer until the moisture content of the material is 1-10% by weight or less to form a dried material, reacting the dried material with a catalyst for a time period sufficient to separate the hydrocarbon and/or polymer from the dried material to form product and recovered material, and collecting the product from the recovered material, the improvement comprising: performing the drying and reacting steps under a slightly negative pressure and substantially in the absence of oxygen.
